(19) United States
(12) Patent Application Publication
(10) Pub. No.: US 2025/0288743 A1
(43) Pub. Date: Sep. 18, 2025

(54) MEDICAL DISPOSABLE MEDICAL DEVICE

(71) Applicant: B. Braun Melsungen AG, Melsungen (DE)

(72) Inventors: Michael Kauba, Fuldabrueck (DE); Markus Vogel, Katlenburg-Lindau (DE); Martin Sielemann, Wolfhagen (DE); Michael Ostermoeller, Bad Hersfeld (DE)

(21) Appl. No.: 19/079,624

(22) Filed: Mar. 14, 2025

(30) Foreign Application Priority Data
Mar. 15, 2024 (EP) 24163941.8

(51) Int. Cl. A61M 5/31; A61M 5/00

(52) U.S. Cl. CPC A61M 5/3129; A61M 5/008; A61M 2205/6009; A61M 2205/6018; A61M 2205/6036; A61M 2205/6072; A61M 2205/6081

(57) ABSTRACT

A disposable medical article for a medical device includes a machine-readable identification feature. The machine-readable identification feature can be a feature of the disposable article or be attached to a predetermined partial section of the disposable medical article in a detachable or non-detachable manner. The disposable medical article can be a syringe for use in syringe pumps.

### CROSS-REFERENCE TO RELATED APPLICATION

This application claims priority under 35 U.S.C. § 119 to European Application No. 24163941.8, filed on Mar. 15, 2024, the content of which is incorporated by reference herein in its entirety.

### FIELD

The present disclosure relates to a disposable medical article for a medical device, in particular a syringe for use in a syringe pump, a medical device, in particular a syringe pump, for the automated administration of medication to a patient, a machine-readable recognition feature for disposable articles in a medical device, a system and a method for recognition feature-dependent, in particular personalized, medication administration.

### BACKGROUND

In the prior art, medical devices, in particular syringe pumps, are known which are used in the therapy of a patient and in particular to administer medication to the patient automatically. Disposable medical articles, such as syringes for use in syringe pumps, are inserted into the medical device, which then delivers the medication contained in the disposable article, such as a syringe for use in syringe pumps, to the patient in a controlled manner. In particular, a drive unit present in the medical device is used, which can be controlled by a corresponding control device.

For example, a medication or other medical solution, such as saline solution, can be provided in a syringe for use in syringe pumps. These accessories containing the medicine can then be inserted into the medical device. The actuator that operates the syringe can then be controlled so that the desired amount of solution is administered to the patient. This is achieved, for example, by the drive unit and, if applicable, a drive head surrounded by it, pressing it onto a syringe plunger plate by advancing a membrane disk and thus pressing the plunger/syringe plunger into the syringe plunger. The resulting pressure causes the desired amount of solution to be released from the syringe opening of a syringe for use in syringe pumps in a controlled manner.

A large number of such medical devices and associated accessories are known in the state of the art.

However, there is generally the problem that in everyday medical practice and possibly in home use, the correct medication reaches the patient via the desired access, in the desired quantity and at the desired time, and the medical system, comprising the medical device and the medical accessory, should be set up in such a way that a mix-up or other inadvertent incorrect operation is excluded as far as possible.

In practice, clinical staff usually select an accessory preloaded with a medical solution and possibly a medication, or load it themselves with a solution and possibly a medication, and then insert the loaded accessory into the medical device. If necessary, the selected accessory can be selected and confirmed on a graphical user interface of the medical device. More modern systems have a corresponding network interface and may communicate with an external control unit.

This is of particular relevance when medical systems, for example in intensive care, comprise a large number of medical devices in order to provide patients with a large number of different medications and solutions at the same time, possibly via a large number of access points. This can result in a large number of medical tubes surrounding the patient and the safety against errors when connecting by medical personnel can be impaired.

If the wrong medication is administered or the wrong delivery rate of the medical device is selected, this can have fatal consequences for the patient. In particular, the administration of medication into the wrong patient access can be immediately highly critical, or especially if low delivery rates are set any existing alarm that would detect an insufficient administration of medication or medical solution can only be triggered late. The mixing of medication and/or solutions that may not be compatible with each other can also occur due to a lack of overview of the routing of various accessories and medical devices.

At present, the identification of suitable accessories is carried out manually, in particular by visually and manually tracing the tube from the medical device to the patient access point. Various solutions are known in the state of the art, which in particular provide for marking of the accessories in order to prevent confusion when fitting the medical device with the accessory.

For example, document WO 23280455 A1 discloses a medical tubing or a medical sliding clamp, for use on or in a medical device, wherein the disposable medical article comprises at least one integrated marker as an identification feature.

It remains problematic to avoid errors when connecting and assigning accessories and medical devices, particularly with regard to use in a multi-component system.

### SUMMARY

It is therefore an underlying task of the present disclosure to overcome the above-mentioned and other disadvantages. It is a further task underlying the present disclosure to increase safety when connecting medical solutions and, if applicable, disposable articles comprising medicaments, in particular syringes for use in syringe pumps, to a medical device as intended and, in particular, to ensure that the respective disposable article is assigned to the correct medical device as intended. In addition, it is an underlying task of the present disclosure to facilitate the intended setting of the medical device, in particular with regard to the delivery of medical solution and possibly medication, which is included in the accessory article, to the patient.

The tasks described at the beginning are solved by a disposable medical article for a medical device, in particular a syringe for use in syringe pumps, a medical device, in particular a syringe pump, for the automated administration of medication to a patient, a machine-readable recognition feature for disposable articles in a medical device, a system and a method for the recognition feature-dependent, in particular personalized, administration of medication.

Preferred embodiments can be taken from the following description, in particular comprising various embodiments as set forth and described.

The skilled person will thereby understand that any embodiment described in the following description is encompassed and included by the subject matter of the present disclosure. The embodiments, features and combinations of features as described herein in connection with the present disclosure, but also any combination of features as mentioned and described in connection with the embodiments, are deemed to be disclosed herein.

In particular, each function and each combination of functions in the present embodiments may, for example, be applied in a different combination, not least because the skilled person will recognize that each individual combination of the functions mentioned herein is suitable for contributing to the solution of the underlying problem.

Further, any function and any combination of functions in the description below may be used independently. For example, in an arbitrary combination selected from the disclosure, one or more of the embodiments described below and/or embodiments presented from the accompanying figures may be contemplated.

In the context of the present application, terms such as “lateral”, “rear”, “front”, “top”, “bottom”, “opposite”, “inside”, “outside” or the like as used herein, which describe the position of a first object relative to another object, preferably refer to the relative position of a respective part or object with respect to its position when it is fully assembled for its intended use. In other words, these terms are preferably used in reference to a disposable article or medical device according to the present disclosure with respect to the relative position of the respective part or object with respect to its position when the disposable article according to the present disclosure is fully assembled for its intended use in the medical device according to the present disclosure.

According to a first aspect of the present disclosure, the present disclosure relates to a disposable medical article for a medical device, in particular syringe pumps, wherein the disposable article is preferably a syringe for use in syringe pumps. A disposable article according to the present disclosure comprises an identification feature, wherein the machine-readable identification feature is a feature of the disposable article or is detachably or non-detachably attached to a predetermined portion of the disposable article, in particular at the factory. A medical device according to the present disclosure is preferably a syringe pump, or a syringe pump pump. A disposable medical device according to the present disclosure is preferably a syringe for use in syringe pumps.

The term “identifying feature” as used herein preferably refers to any feature capable of distinguishing or identifying an intended disposable article according to the present disclosure from others. In other words, a machine-readable identifier is a characteristic element, feature or marking that serves to distinguish or identify an intended disposable article according to the present disclosure from others.

In various contexts, a machine-readable identifier can be represented by visual, technological, physical or other characteristics. This feature enables clear identification or differentiation in the context of a specific system, application or task. Examples of identifiers include color codes, barcodes, QR codes, biometric features, RFID tags, or other characteristic features used for identification as described herein.

In particular, an RFID tag has the advantage that it can store and carry more complex data that goes beyond the pure identification of the disposable item. For example, an RFID tag can contain information about the patient and/or applications. It is also possible to write application information back onto the RFID tag. This can also ensure, for example, that the exact expiration date of the item is not exceeded.

The disposable article can be manufactured in one or more parts. The machine-readable identification feature is intended to mark the disposable article in such a way that it can be distinguished from other disposable articles or identified using the identification feature. This is done in particular by marking the disposable article by means of the recognition feature on a predetermined section of the disposable article. According to the present disclosure, the machine-readable identification feature can be detachably or non-detachably attached to the disposable article, in particular at the factory. The machine-readable identification feature can additionally or alternatively also be a feature of the disposable article itself, e.g. the color of the syringe. An identification feature that is “detachably or non-detachably attached to a predetermined section of the disposable article, in particular at the factory” can preferably additionally or alternatively also be understood as a feature that is an integral feature of the section or part of the disposable article, e.g. the color of the syringe plunger.

It is possible, for example, to make a disposable item identifiable at the factory by permanently attaching the identification feature. Such a feature can be provided on site, for example molded on or formed in a mold as an integral part. Alternatively, the identification feature can be printed on. Alternatively or additionally, the disposable article can be provided with a machine-readable identification feature manually, e.g. by hospital staff, either detachably or non-detachably. For example, the desired marking and individualization can be achieved by clipping on, attaching or attaching a feature in the form of or with the help of a fastening means, e.g. a rubber sleeve. In particular, the subsequent attachment of an identification feature before use, especially at the factory, allows targeted and user-defined individualization. However, a syringe plunger or a syringe plunger plate in particular can also be printed and provided with an identification feature at the factory. Alternatively, a recognition feature can also be clipped or attached to a syringe plunger plate in particular.

The present disclosure and the disposable medical article, in particular in the form of a syringe for use in syringe pumps, with the identification feature of the disposable article allows particularly advantageous therapy sets and the medication to be assigned, in particular preconfigured, in relation to the application and/or patient. This offers particular advantages in terms of increased safety and may allow less trained personnel, e.g. private individuals in the home care sector, to use the single-use article safely in the medical device.

The machine-readable identification feature, in particular the predetermined portion, is preferably selected so that it can be read by a medical device, preferably in an inserted state. The predetermined portion is selected from the group comprising syringe barrel wing, plunger and/or syringe plunger and/or syringe plunger plate, preferably of a syringe for use in syringe pumps.

In an advantageous embodiment of the disposable article according to the present disclosure, the predetermined portion is a part that faces a drive head, in particular a membrane disk, of a medical device, in particular a syringe plunger plate. This specific orientation facilitates the precise identification and application of the disposable article by the medical device at a particularly accessible location, since the drive head, and in particular the membrane disk, come into contact with the disposable article, namely the syringe plunger plate, as intended in order to push the syringe plunger into the syringe barrel.

The term “in an inserted state” as used herein preferably refers to the correct position of the disposable article in the medical device for the intended use, i.e. when, for example, the syringe is correctly inserted and held in the medical syringe pump for use in syringe pumps.

In a further advantageous embodiment of the disposable article according to the present disclosure, the machine-readable identification feature is selected from a shape-based coding, in particular a 3D structure,
an electronic-physical measurand, in particular a conductivity, a magnetization and/or a wireless wave, in particular an RFID tag, and/or
an optical code, in particular a color and/or a pattern, a barcode or QR code.

The recognition feature is particularly advantageous in the form of a shape-based coding, in particular a 3D structure. This shape-based coding can be characterized by various features such as shaping, relief, corrugation and the like. The molding refers, for example, to the specific design or contour of an area on the disposable article that serves as a distinguishing feature. The relief describes, for example, elevations or depressions on the surface of the disposable article that form a specific pattern or texture. Corrugation, for example, refers to regular elevations, often in the form of grooves or waves, which are distributed over the surface.

By integrating this shape-based coding, in particular as a three-dimensional structure, into the predetermined section of the disposable article, a unique and tactilely detectable coding is created. This enables reliable identification of the disposable article by touch or tactile detection, which is particularly advantageous in environments where visual identification methods are limited.

Additionally or alternatively, the recognition feature is designed as an electronic-physical measured variable, in particular a conductivity, a magnetization and/or a wireless wave, in particular an RFID tag. The recognition feature as an electronic-physical measurand can additionally or alternatively comprise a “miniature shield”. A miniature shield preferably refers to an expansion board or component that can be attached and/or plugged onto a microcontroller board in particular. The term “miniature shield” should preferably mean that it is a compact or small version of such an expansion module. This can be advantageous with the small space or weight requirements for realizing the present disclosure. According to the present disclosure, a miniature shield can contain a part of the sensor arrangement, in particular sensors, but also communication modules or other components in order to extend the functionality of the microcontroller.

Conductivity preferably refers to the ability of the disposable article to conduct electrical current, which can be used as a measurable physical property. Magnetization refers, for example, to the alignment of magnetic moments in the material of the disposable item, which in turn can represent a characteristic measured variable. Wireless waves, in particular RFID tags, preferably enable wireless identification and communication via electromagnetic waves. By integrating such electronic-physical measured variables into the predetermined section of the disposable article, automated and precise identification is advantageously made possible. This is particularly useful for fast and contactless identification processes in various applications, such as medical technology.

Additionally or alternatively, the machine-readable identification feature is designed as an optical code, in particular a color and/or a pattern, a barcode or QR code. The color can comprise both black and white as well as different colors. The color represents a visually perceptible characteristic of the disposable item and/or predetermined section of the disposable item. A pattern can contain additional visual elements that serve as characteristic features for identification. The barcode and QR code are specific optical codes that are machine-readable by scanning or photographic capture and contain information about the disposable article. This integration of optical codes enables efficient and precise visual identification of the disposable item, especially in applications based on optical recognition, such as when using scanners or cameras. In particular, an illuminated RFID reader can be used as a recognition feature. Many of these reader modules have a control ring that can be used for signaling. This helps to further increase application security. The illumination during the reading process improves readability and ensures that identification is reliable. However, this concept can generally be applied to any identification method in order to optimize and secure user guidance. It helps to ensure intuitive user guidance and to make the identification of disposable items efficient and reliable. A ring illuminated in this way can be used together with or independently of an RFID tag.

In a second aspect of the present disclosure, the present disclosure relates to a medical device, in particular a syringe pump, for the automated administration of medication to a patient. A medical device according to the present disclosure comprises
a recognition device for identifying an assignable, classifiable and/or personalizable disposable article, comprising a sensor arrangement which is set up to detect the machine-readable recognition feature of the disposable article;
a control device which is connected to the sensor arrangement and identifies the machine-readable recognition feature of the disposable article on the basis of the information recorded;
a drive unit controlled by the control device to automatically administer the medication to the patient according to the recognized information of the disposable article,
   wherein the disposable article is preferably a disposable article according to one of the preceding aspects.

A recognition device for identifying an assignable, classifiable and/or personalizable disposable article comprises a sensor arrangement that is specifically designed to detect the machine-readable recognition feature of the disposable article. This sensor arrangement can comprise various technologies and sensors aimed at precisely registering the defined recognition feature of the disposable article. A control device, which is connected to the sensor arrangement, takes over the processing of the captured information in order to identify the machine-readable recognition feature of the disposable article. The control device preferably acts as the central processing element and uses the data provided by the sensor arrangement to uniquely identify the disposable article. The connection between the sensor arrangement and the control device enables a seamless exchange of information, allowing the captured data to be analyzed efficiently and precisely. A processing device is preferably provided between the sensor arrangement and the control device, which processes the information recorded by the sensor and, if necessary, retrieves associated data from a memory unit

Based on this analysis, the control device identifies the specific recognition feature that is assigned to the assignable, classifiable and/or personalizable single-use article. This identification forms the basis for the further steps in the application, be it in drug administration or another context in which the disposable is used. A drive unit is controlled by the control device in order to automatically administer the medication to the patient according to the recognized information of the disposable article. The control device therefore orchestrates the functions of the drive unit based on the identified information of the disposable article. The drive unit may include various mechanical or electronic mechanisms that are used to deliver the medication to the patient according to the specific requirements and dosages. In particular, the guidance of the syringe plunger of a syringe for use in syringe pumps within the syringe barrel by means of a membrane disk moved by a drive head is known in the prior art. Through integration with the control device, the administration of the medication is automated and precisely controlled, thereby improving the safety and effectiveness of medical treatment.

In an advantageous embodiment of the medical device according to the present disclosure, it comprises an activation device for activating and deactivating the detection device. This is advantageous in that the detection device can be activated or deactivated as required. In other words, the detection device can be deactivated when it is not needed. This can, for example, help to save energy and extend battery life. If necessary, the activation device can activate the detection device to enable the identification of disposable items again. This approach improves the energy efficiency of the medical device and ensures that the recognition device is only in operation when it is actually needed, which can be a practical advantage, especially in battery-powered devices. For activation and/or deactivation, the medical device can have a corresponding actuating element, in particular in a display.

In a further advantageous embodiment of the medical device according to the present disclosure, the device comprises a communication interface for transmitting the captured information to external control devices or networks. A significant advantage of this configuration is that the captured information can be shared with external devices or networks via the communication interface. This enables seamless integration of the medical device into wider medical systems or data platforms. External control devices can use the information received, to control additional functions or to make higher-level decisions. Transmission to networks enables remote monitoring and control of the medical device, which is particularly advantageous in extensive medical infrastructures. This makes it particularly advantageous to integrate the device according to the present disclosure into a system according to the present disclosure, so that, for example, a cloud-based application database, which preferably contains information on components of the system, including information on the pump, patient ID, treatment regimens and medication details, can be addressed by the medical device via the communication interface and recorded information can be transmitted or compared with information from the cloud-based application database.

In a further advantageous embodiment of the medical device according to the present disclosure, the drive unit, in particular a drive head or membrane disk of the drive unit, comprises the detection device.

Preferably, the detection device is designed as part of the drive unit, in particular a drive head of the drive unit, and/or is attached to the drive unit, in particular a drive head of the drive unit.

The drive unit, in particular a drive head or membrane disk, preferably comprises the detection device and/or the detection device is also preferably designed as part of the drive unit, in particular a drive head of the drive unit, and/or the detection device is also preferably attached to the drive unit, in particular a drive head of the drive unit.

This arrangement offers the advantage that the detection device is integrated directly into the drive unit. This results in a compact design and improved functionality. The recognition device can thus work efficiently with the drive mechanism and capture precise information about the assignable, classifiable and/or personalizable disposable article. This is particularly the case if components of the disposable medical article, e.g. a syringe plunger plate, bear the recognition feature, which then comes close to a component of the drive unit as intended or comes into contact with it, e.g. with a membrane disk. This contributes to a safe and reliable detection of the detection feature by the detection device by ensuring a direct connection between the drive unit with detection device and the detection feature on a component of the disposable article driven by the drive unit.

In a further advantageous embodiment of the medical device according to the present disclosure, the medical device has a support mechanism, in particular a clamping mechanism, for supporting the disposable article in the medical device. The support mechanism comprises the recognition device. Preferably, the detection device is attached to a support mechanism, in particular a clamping mechanism, for supporting the disposable article in the medical device and/or is designed as part of a support mechanism, in particular a clamping mechanism, for supporting the disposable article in the medical device. This allows, for example, the detection of a recognition feature provided on a component of the disposable article intended for the holder. For example, the recognition of a recognition feature attached to the syringe barrel wing can be achieved by a recognition device on a support mechanism, in particular a clamping mechanism, for supporting the disposable article in the medical device.

In a further advantageous embodiment of the medical device according to the present disclosure, the drive unit, in particular a drive head or membrane disk, has a sensor signal opening. The detection signals of the sensor arrangement can reach the detection feature through such a sensor signal opening. For example, it can be achieved that the sensor signals from the sensor arrangement can detect the machine-readable recognition feature. In particular, the sensor signal opening can be formed by a material that is compatible with the sensor signal, in particular a transparent material, an integrated window, an optical lens or a material cut-out and/or material recess. If such a sensor signal opening is arranged, for example, in a membrane disk, the measurement can, for example, be carried out particularly advantageously through the membrane disk and the sensor arrangement can be provided at a different location, in particular behind the membrane disk in the drive head. An integrated optical lens can also focus or amplify the signal.

In a further advantageous embodiment of the medical device according to the present disclosure, the detection device comprises an optical conductor, preferably an optical fiber, connected to the sensor arrangement and preferably flexibly shaped. This has the advantage that the optical conductor, preferably light guide, on the one hand enables a more precise and flexible detection of the machine-readable recognition feature and on the other hand can guide light from a light source surrounded by the sensor arrangement in order to illuminate the recognition feature if necessary and make it easier to detect. Due to the flexibility of the optical guide or light guide, the sensor arrangement can be efficiently adapted and arranged to different geometries and shapes of the assignable, classifiable and/or personalizable disposable article and within the medical device. This contributes to the versatility of the detection device and ensures reliable identification of the disposable article. The flexible optical conductor, in particular the light guide, also enables optimum light guidance, which further improves the accuracy of the detection. The guidance of information through such a flexible optical conductor can be particularly advantageous when using an optical code, especially a color and/or a pattern, a barcode or QR code. The design of a membrane disk with translucent material is particularly advantageous, whereby a corresponding sensor of the sensor arrangement can be coupled directly to the membrane disk or coupled indirectly, and whereby the measurement signal can then be guided through an optical conductor, e.g. an optical fiber or similar. Furthermore, the membrane disk can have an optical lens or an optical window. In this embodiment, too, a corresponding sensor of the sensor arrangement can be coupled directly to the membrane disk or coupled indirectly, and the measurement signal can then be guided through an optical conductor, for example an optical fiber or the like.

In a further advantageous embodiment of the medical device according to the present disclosure, the detection device is set up for centric detection of the detection feature, in particular a detection feature on a syringe plunger plate. The precise alignment of the sensor arrangement, so that a detection feature provided on a syringe plunger plate can be detected centered, can be advantageous in detecting the information provided by the detection feature and the detection feature itself. In other words, the sensor arrangement is set up and aligned in such a way that it can optimally detect a provided detection feature.

In a further advantageous embodiment of the medical device according to the present disclosure, the device and in particular the detection device comprises a shading device for at least partially shading the detection feature. It is preferred that the shading device is designed as part of a support mechanism, in particular a clamping mechanism, for supporting the disposable article in the medical device. In particular in embodiments in which an optical code, in particular a color and/or a pattern, a barcode or QR code, is used as a recognition feature, it can be advantageous to shade and/or darken the area of the recognition feature and, if necessary, to illuminate it with a light source. In this way, the effect of ambient light can be reduced and the illumination can be optimized using the system's own light source. This can be provided particularly advantageously by attaching a shading device to existing components of a medical device. For example, in a syringe pump for a syringe for use in syringe pumps, a circumferential syringe plunger gripping mechanism can be designed so that it can be modified and used as a shading device.

In a further advantageous embodiment of the medical device according to the present disclosure, the sensor arrangement is suitable for detecting the machine-readable recognition feature. Such a sensor arrangement can in particular be selected from a sensor arrangement for detecting a shape-based coding, in particular a 3D structure. This can include, for example, a resistive touch sensor and/or a scanning mechanism.

Such a sensor arrangement can in particular also be selected from a sensor arrangement for detecting an electronic-physical measured variable, in particular conductivity, magnetization and/or a wireless wave, in particular an RFID tag. In particular, conductivity sensors, for measuring electrical resistance or conductivity; magnetic field sensors, for detecting changes in the magnetic field that may indicate magnetization; or Hall effect sensors, for detecting changes in the magnetic field based on the Hall effect; or RFID sensors, for reading and interpreting the data transmitted by RFID tags via radio waves, can be used here.

Such a sensor arrangement can be selected in particular from a sensor arrangement for detecting an optical code, in particular a color and/or a pattern, a barcode or QR code. For this purpose, for example, color or code recognition can be provided by means of a code scanner and/or a camera or similar. In the case of color recognition, it is important to set the lighting of the recognition feature as optimally as possible and, if necessary, to protect it from the effects of daylight in order to enable better reading. A shading device can be advantageously combined for this purpose. Due to the freedom of orientation, the use of a QR code in particular allows a high level of recognition reliability.

The person skilled in the art will be able to design the sensor device in such a way that the sensor is optimally positioned in relation to the detection feature and thus, for example, a distance between the sensor and the detection feature is selected that allows good feature detection.

In a particularly preferred embodiment, the syringe plunger plate of a syringe for use in syringe pumps is printed with a QR code as an identification feature. Alternatively, a QR code can also be provided as a clip-on and/or stick-on solution on the syringe plunger plate.

The selection of the sensors mentioned herein depends on the requirements of the specific application and the selected detection features, and different technologies may be preferred depending on the context. The sensors mentioned can also be combined in the sensor arrangement in order to detect several measured variables simultaneously or to ensure redundant detection.

In a further embodiment of the medical device, the control device is arranged to be able to instruct the drive unit to stop or modify the administration of the medication, in particular if a discrepancy or an error is detected in the identification of the assignable, classifiable and/or personalizable disposable article.

In a further embodiment of the medical device, the control device is set up to activate an alarm function if the captured machine-readable identification feature of the disposable article does not match predefined information or if an error occurs during identification.

According to a third aspect of the present disclosure, the present disclosure relates to a machine-readable identification feature for disposable articles in a medical device, in particular a syringe pump. A machine-readable identification feature according to the present disclosure is provided such that it is suitable for shape-based coding, in particular a 3D structure, for coding by means of an electronic-physical measurand, in particular a conductivity, a magnetization and/or a wireless wave, in particular an RFID tag, and/or for coding by means of an optical code, in particular a color and/or a pattern, a barcode or QR code.

A machine-readable identification feature for disposable articles according to the present disclosure according to the third aspect is preferably a machine-readable identification feature for a disposable article according to the present disclosure and according to the first aspect. A machine-readable identifier for disposable articles according to the present disclosure according to the third aspect is preferably a machine-readable identifier for a disposable article for use in a medical device according to the present disclosure and according to the second aspect.

A recognition feature according to the present disclosure is preferably shaped such that it can be detachably or non-detachably attached to a predetermined portion of a disposable article. This allows a disposable article to be preconfigured by means of the identification feature, for example a syringe for use in syringe pumps with the aid of an attachable or hinged miniature shield or other identification features until immediately before use. In particular, a recognition feature according to the present disclosure can also be used to individualize disposable articles that are available as mass-produced articles and are not specifically intended for use in a system according to the present disclosure.

According to a fourth aspect of the present disclosure, the present disclosure relates to a system for recognition feature-dependent, in particular personalized, drug administration, in particular for medical devices such as syringe pumps, comprising: a memory unit which preferably comprises information on components of the system and/or a drug database in which data on drugs are stored and/or a patient database in which data on the patient are stored.

The storage unit can be provided locally in the device and/or outside the device, whereby a storage unit provided outside the device comprises in particular a network, server and/or cloud-based storage unit.

The system further comprises a medical device, preferably according to the present disclosure, which has a communication interface for transmitting the acquired information. The communication interface is preferably provided and arranged to transmit the acquired information to a storage unit and/or to retrieve data from the storage unit. In particular, such a communication interface can be set up to communicate with a storage unit provided outside the device, in particular a network-, server and/or cloud-based storage unit. Such a communication interface can include communication and server protocols and/or also have a LAN, WLAN or other wired and/or wireless interfaces.

The system further comprises a disposable medical article for a medical device, in particular a syringe pump, wherein the disposable article is in particular a syringe for use in syringe pumps, and comprises a machine-readable identification feature which is a feature of the disposable article or which is detachably or non-detachably attached to a predetermined portion of the disposable article, in particular at the factory.

In a particularly preferred embodiment of the system according to the present disclosure, the control unit is provided and set up to filter and/or limit a drug selection option in the drug database on the basis of the detected and thus identified disposable article; and/or to filter and/or limit a selection option of a further disposable article; and/or to issue an alarm and/or prevent the start of therapy when a therapy of the medical device with a predetermined combination of drug or drug concentration to disposable article is set.

In a particularly preferred embodiment of the system according to the present disclosure, the control unit is provided and set up to set a function mode of the pump, e.g. the delivery speed of a medication and/or the feed speed of a syringe plunger; restriction of functions, restriction of the medication database or the like, on the basis of the detected and thus identified disposable article.

The memory unit preferably comprises information on components of the system, e.g. information on the medical device, e.g. on the pump, pump performance, pump specification or other device features.

At the same time or alternatively, the storage unit preferably comprises a patient database in which patient data is stored, e.g. a patient ID, treatment regimens and medication details, or the like. Further information may also be in the patient database, in particular personal and/or health-related patient data, such as age, weight, name, etc.

At the same time or alternatively, the storage unit preferably comprises a medication database in which medication data is stored, e.g. dosage regimens, expiration dates or similar.

The communication interface preferably transmits information recorded in the system according to the present disclosure to a storage unit and/or retrieves data from the storage unit. The retrieval of data is preferably dependent on the medical disposable item for a medical device recognized by means of the machine-readable recognition feature, which is transmitted from the communication interface to the storage unit.

In this context, the disposable article and/or the medical device and/or the machine-readable identification feature is preferably a disposable article and/or a medical device and/or a machine-readable identification feature according to the present disclosure.

In a system according to the present disclosure, the recognition and assignment of assignable, classifiable and/or personalizable disposable articles is carried out by a specific machine-readable recognition feature, which is recognized by the recognition device of the medical device. Based on this recognition and assignment and the disposable article, predetermined information can be assigned to the disposable article in particular, which was retrieved from the memory unit based on the recognition of the recognition feature.

The storage unit preferably comprises information on components of the system and/or a medication database in which medication data is stored and/or a patient database in which patient data is stored

Recognition of the unique identifier by the medical device allows unique retrieval of predetermined information from the cloud-based application database based on the unique identifier. With such information and based on the unique assignment of the unique item, for example, a patient ID, treatment regimens and medication details can be retrieved from the cloud-based application database, which may be necessary and/or helpful for the safe treatment of the patient.

At the same time, information on system components, the pump, safety features such as time, date, serial number and pump can be used to prevent incorrect therapy on the patient.

In addition to retrieving information from the cloud-based application database, a device and/or system according to the present disclosure can also be set up and/or provided to write information, for example therapy, medication, system, device or patient information, to the cloud-based application database. For example, once the inserted disposable article has been recognized, this disposable article can be assigned to a specific patient and/or a specific patient ID in the cloud-based application database. Alternatively or additionally, information about the disposable article, for example a usage date, expiration date, remaining volume or similar, can be assigned to the identified disposable article in the cloud-based application database.

The local data synchronization between the medical device and the cloud-based application database advantageously ensures the consistency of the information and the assignment of the disposable item uniquely identified by the recognition feature. Together with the information about the medical device, all system components can thus be clearly assigned to each other and possibly also to the patient.

Based on such a system according to the present disclosure, various embodiments according to the present disclosure are possible, which are covered by the present disclosure. In particular, a cloud-based database with optional retrieval at the device.

In particular, a cloud database can include applications, patient information, treatment regimens and medication details as predefined data and transmit these to the medical device via a network connection, possibly wirelessly. In particular, such a transfer can be triggered or enabled by the recognition of the recognition feature of a disposable item. The assignable, classifiable and/or personalizable disposable item can then also include predefined information, e.g. a patient ID, a medication and details about therapy and treatment regimen. This data can be assigned to the medical device by recognizing the disposable item using the recognition feature and transmitted to it or read out by it using the recognition device. A data comparison of information from the cloud database and information from the assignable, classifiable and/or personalizable disposable item, as well as any other information that can be entered manually, can then be compared. Such a comparison can take place locally on the medical device and/or in a corresponding server or cloud application. In particular, the medical device can compare local data with the cloud and assign the assignable, classifiable and/or personalizable disposable item to the patient information.

In another embodiment, a cloud-based database with key code transmission may be provided. Thus, a key code can be generated in a cloud application and transmitted to the medical device. The key code may be generated using various predefined information, in particular information about the medical device, for example a serial number, or information about the patient, in particular a unique patient ID, information about medication and/or treatment regimen. The key code can then be issued to the medical device. The medical device can be set up in such a way that only a disposable item that matches the key code is accepted. An assignable, classifiable and/or personalizable disposable article can also contain predefined information, e.g. on an identification feature, or be assigned such information after the disposable article has been identified. In this way, the assignable, classifiable and/or personalizable disposable item can be assigned to various predefined data locally or in the cloud application. The machine-readable identification feature of the disposable item is then matched with the key code to ensure acceptance by the medical device.

A cloud-based database with over-the-air data synchronization can also be provided. The cloud database again contains predefined information, including medical device information, patient information and a patient ID and treatment details. After inserting the disposable item into the medical device, it is recognized by the recognition feature and assigned to the device. On the one hand, the medical device can compare the data in the cloud database, and on the other hand, a key code generated by the cloud can be retrieved, which can then be entered manually in the medical device for confirmation. The data synchronization between the cloud and the medical device can preferably be carried out wirelessly. By entering a key code manually or via the cloud to the medical device, the machine-readable identification feature of the disposable item can be verified.

In one embodiment, the system according to the present disclosure is preferably set up in such a way that the recognition device detects assigned, classified and/or personalized features of the disposable article and ensures that only disposable articles whose features match the data in the cloud are accepted.

In one embodiment, the system according to the present disclosure is preferably set up in such a way that the recognition device ensures that the local data comparison is only carried out if the personalized disposable article is successfully checked against the associated key code.

In one embodiment, the system according to the present disclosure is set up in such a way that the cloud-based application database contains further information such as application time and interval and this information is included in the local data synchronization to ensure precise and personalized drug administration.

According to a fifth aspect of the present disclosure, the present disclosure relates to a method for personalized drug administration in a medical system, in particular for syringe pumps. A method according to the present disclosure comprises at least the following steps:
providing a medical device, preferably a device according to one of the preceding aspects,
an insertion of a disposable article for the medical device, preferably a disposable article according to one of the preceding aspects, comprising a machine-readable identification feature, wherein the machine-readable identification feature is a feature of the disposable article or is attached to a predetermined portion of the disposable article in a detachable or non-detachable manner, in particular at the factory;
recognizing the disposable article, preferably by means of a recognition device of the medical device, by means of the machine-readable recognition feature of the disposable article;
identification of the recognition feature of the disposable item based on the information recorded;
controlling a drive unit, preferably by means of a control device of the medical device, for the automated administration of a medicament according to recognized information of the disposable article.

It should be noted that the steps listed above do not necessarily have to be carried out in the order given. The steps provided can be carried out in any other suitable order.

However, the above order may apply to certain embodiments of the method.

It will be immediately recognized by a person skilled in the art that a property, embodiment, effect or advantage described herein in connection with the inventive disposable medical article for a medical device, in particular a syringe for use in a syringe pump, the medical device, in particular a syringe pump, for the automated administration of medicaments to a patient, the machine-readable identification feature for disposable articles in a medical device, and the system for the identification feature-dependent, in particular personalized, administration of medicaments, can also be a property, embodiment, effect or advantage of the inventive method, and vice versa.

In one embodiment, the method according to the present disclosure may further comprise matching predefined information identified using a recognition feature of the disposable article with predefined information in a cloud-based application database.

In one embodiment, the method according to the present disclosure may further comprise ensuring, by means of a recognition device, that the local data comparison is only carried out if the personalized disposable article is successfully checked against the associated key code.

In one embodiment, the method according to the present disclosure may further comprise that the cloud-based application database contains additional information such as application time and interval and this information is included in the local data reconciliation to ensure precise and personalized medication administration.

All described embodiments of the present disclosure have the advantage that there are significant improvements both in the short term and in the medium to long term compared to the solutions known in the prior art. The recognition feature particularly advantageously increases safety in the selection and use of medical devices due to the possibility of pre-parameterization. In addition, the present disclosure involves only a relatively small additional cost for the disposable article. The present disclosure also enables integration into various fields of application such as homecare, since the avoidance of misassignment of medication to patients is avoided, and thus untrained personnel can more easily use the present disclosure. Furthermore, predefined information can be transferred more easily, for example, the transfer of therapy patterns to different medical devices allows the medical device to be exchanged easily. The possible cloud connection also ensures secure and efficient data transmission, regardless of the location distance. Overall, the security features help to ensure correct identification of personalized disposables and thus increase patient safety.

### BRIEF DESCRIPTION OF THE DRAWINGS

The present disclosure is explained in more detail below with reference to the drawings, from which further features, embodiments and advantages can be seen, and in which:

FIGS. 1A, 1B, 1C, and 1D show schematic representations of embodiments of disposable articles and identifying features according to the present disclosure; and

FIG. 2 shows a schematic representation of an embodiment of a medical device according to the present disclosure.

The features of the present disclosure, as disclosed in the description, examples and/or figures, may be used individually or in any combination.

In the embodiments shown in the figures, elements that are similar or identical in function are designated with the same reference symbols. It should be noted that the figures may not be true to scale.

### DETAILED DESCRIPTION

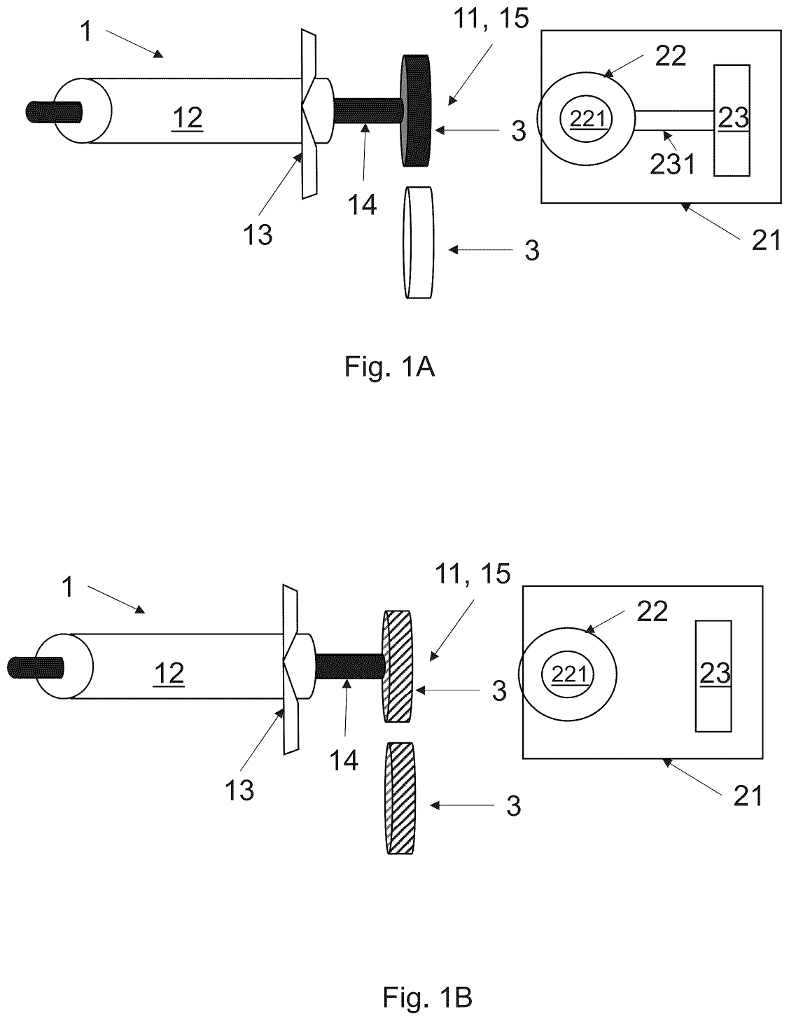

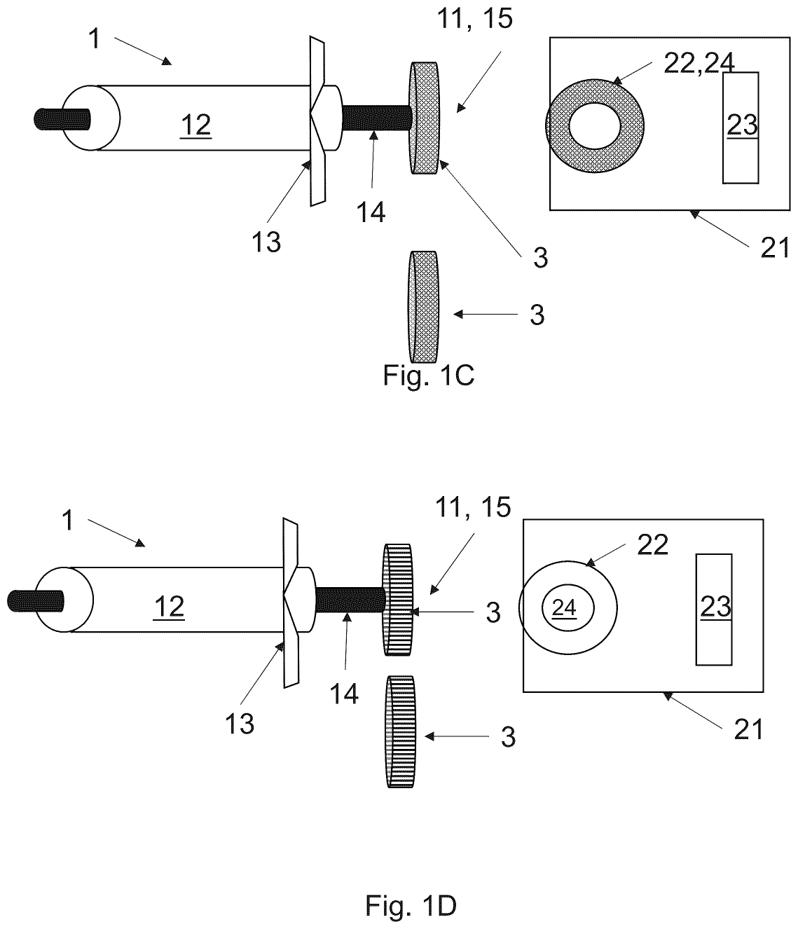

FIG. 1A shows a schematic representation of a first embodiment of a disposable medical article 1 for a medical device 2, in the form of a syringe pump 2. The disposable article 1 is shown as a syringe for use in syringe pumps 2 and comprises a machine-readable identification feature 3. The syringe for use in syringe pumps 1 comprises a syringe barrel 12 in which the medication to be administered is contained. For manual use, tip cylinder wings 13 are usually formed on the cylinder 12 to facilitate manual administration of a medication with the syringe pump 2, for example with a cannula attached. These tip cylinder wings 13 also serve to hold and clamp the syringe plunger 14 in the syringe pump 2.

A plunger 14, in the form of a syringe plunger 14, is inserted into the syringe cylinder 12, at the end of which a syringe plunger plate 15 is formed. When the syringe plunger plate 15 is pressed as intended, the syringe plunger 14 is moved in the cylinder 12 and the medication is pressed out of the syringe 1 for use in syringe pumps 2. In a medical device such as a syringe pump 2, a drive unit 21, in particular a drive head 21, is usually provided, which is pressed onto the syringe plunger plate 15 of the inserted syringe for use in syringe pumps 2 in order to apply pressure to the syringe plunger plate 15. In the embodiment shown, the identification feature 3 is a rubber grommet 3 which can be pulled over the syringe plunger plate 15 and is colored. This machine-readable identification feature 3 can therefore be applied to a predetermined section 11, in this case the syringe plunger plate 15, of the disposable article 1. Alternatively, the entire disposable article 1, or a part thereof, for example the syringe plunger plate 15 itself, could also have such a machine-readable identification feature 3, for example in the form of a coloured disposable article 1 or a coloured syringe plunger plate 15. This would result in a feature 3 that is inseparably connected to the syringe plunger plate 15. In particular, this may already be provided at the factory. In the embodiment shown, the predetermined partial section 11, i.e. the syringe plunger plate 15, faces the drive head 21 and the membrane disk 22 of the syringe pump 2.

FIG. 1A also schematically shows the drive head 21 of a syringe pump 2. A recognition device 23 can be seen, which is provided for identifying the personalized disposable syringe for use in syringe pumps 2. The recognition device 23 comprises a sensor arrangement 24, not shown, which is set up to detect the machine-readable identification feature 3 of the disposable article 1.

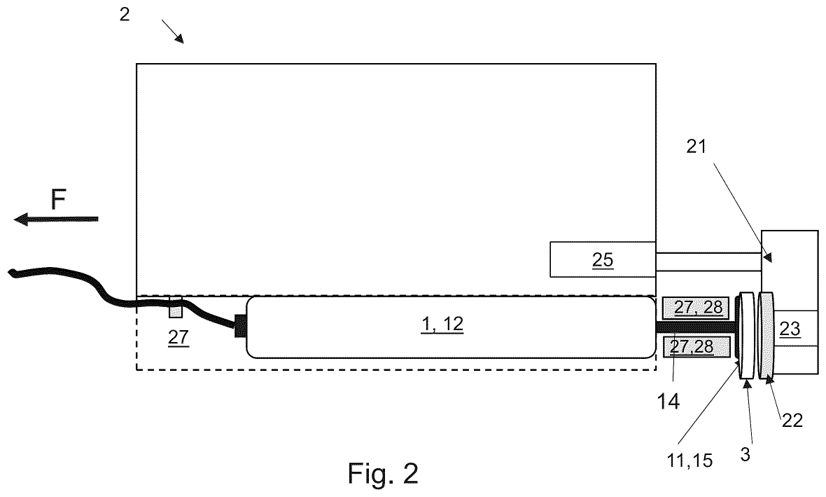

FIG. 2 shows a schematic representation of an embodiment of a syringe pump 2 according to the present disclosure, to which reference is also made below. There, the syringe 1 is inserted into the syringe pump 2. The syringe pump 2 comprises a control device 25, which is connected to the sensor arrangement and identifies the machine-readable recognition feature 3 of the disposable article 1 on the basis of the acquired information. The syringe pump 2 further comprises a drive unit 21, which is controlled by the control device 25 in order to automatically administer the medication to the patient in accordance with the recognized information of the disposable article 1. The drive unit 21, in particular the drive head 21 or diaphragm disk 22, comprises the recognition device 23 as part of the drive unit 21, in particular the drive head 21.

The syringe pump 2 also comprises a support mechanism 27, in particular a clamping mechanism which holds the syringe for use in syringe pumps 2 in the syringe pump 2. In particular, the syringe barrel wings 13 are clamped to the syringe barrel 12. This support mechanism can 27 comprise the detection device 23, but in the embodiment shown in FIG. 2 it serves as a shading device 28 for at least partially shading the detection feature 3, which is shown in FIG. 2 mounted on the syringe plunger plate 15. Here, the shading device 28 is thus designed as part of the support mechanism 27 for supporting the syringe 1 in the syringe pump 2. Alternatively, for example, a clamping mechanism for the syringe barrel wings 13 on the syringe barrel 12 can also be set up as a shading device.

In FIG. 1A, the membrane disk 22 has a sensor signal opening 221. This can be formed in particular by a sensor signal-permeable, in particular transparent material, by an integrated window, an optical lens or a material omission and/or material recess. Here, the detection device 23 is connected to the sensor arrangement 24 and has a flexibly shaped optical conductor, in this case an optical fiber 231, which conducts the detected signal to a detection device 23. This detection device 23 also has a light source which guides the light through the light guide 231 and the opening 221 specifically to the detection feature 3. Thus the detection feature is optimally illuminated in particular also by the shading 28 and the sensor arrangement 24, for example comprising in this case a color detection camera, can optimally read the detection feature. The detection device 23 is arranged in such a way that it is suitable for centric detection of the detection feature 3 on the syringe plunger plate 15.

As an alternative to the flexible light guide 231, an optical system could also be provided that has a cable-connected LED. In this case, the LED can be attached directly to the membrane disk 22.

FIG. 1B shows a further embodiment of the present disclosure. Here, the recognition feature 3 is provided as a shape-based coding, in particular as a corrugated 3D structure either on the syringe plunger plate 15 or as a rubber sleeve. Accordingly, the recognition device 23 comprises another sensor system 24, for example, in the form of a resistive touch sensor 24 or a mechanical pick-up sensor 24. The membrane disk 22 can already have such a sensor system 24 in the opening 221, which can then transmit the signals to a corresponding electronic recognition device 23.

FIG. 1C shows a further embodiment of the present disclosure. Here, the recognition feature 3 is designed as an RFID antenna unit running around the syringe plunger plate 15. This in turn can be permanently mounted or designed as a rubber grommet 3 that can be pulled over or as a plunger plate 15 that can be mounted on/off. Accordingly, the detection device 23 comprises another sensor system 24, e.g. in the form of an RFID antenna unit 24. The membrane disk 22, which is solid here, can have such an RFID antenna unit 24, e.g. as a circumferential design, which can then transmit the signals to a corresponding electronic detection device 23.

FIG. 1D shows a further embodiment of the present disclosure. Here, the recognition feature 3 is designed as a QR code, which is either incorporated into the syringe plunger plate 15 or printed on the coverable rubber sleeve 3. Accordingly, the recognition device 23 comprises another sensor system 24, e.g. in the form of a QR code reader 24. The membrane disk 22 can have such a QR reader unit 24, e.g. in a recess 221, which can then forward the signals to a corresponding electronic recognition device 23.

As shown in FIGS. 1A, 1B, 1C and 1D, the medical device 2 according to the present disclosure can thus be provided, wherein the sensor arrangement 24 is adapted to detect the machine-readable identifier 3 and in particular is selected from a sensor arrangement 24 for detecting:
a shape-based coding, in particular a 3D structure,
an electronic-physical measurand, in particular a conductivity, a magnetization and/or a wireless wave, in particular an RFID tag, and/or a optical code, in particular a color and/or a pattern, a barcode or QR code.

The embodiments illustrated in the figures may relate to preferred embodiments, while all elements and features described in connection with the embodiments, where appropriate, may be used in combination with any other embodiment and feature as discussed herein, particularly with respect to any other embodiment previously discussed.

### LIST OF REFERENCE SYMBOLS

1 medical disposable
11 Subsection
12 Syringe barrel
13 Syringe barrel wings
14 Plunger/syringe plunger
15 Syringe plunger plate
2 Medical device
21 Drive unit, drive head
22 Membrane disk
221 Sensor signal aperture
23 Recognition device
231 Optical conductor
24 Sensor arrangement
25 Control unit
26 Communication interface
27 Support mechanism
28 Shading device
3 Recognition feature
4 System

1. A disposable article for a medical device, the disposable article comprising a machine-readable identification feature, the machine-readable identification feature being a feature of the disposable article or is attached to a predetermined portion of the disposable article in a detachable or non-detachable manner.

2. The disposable article according to claim 1, wherein the disposable article is a syringe for use in a syringe pump.

3. The disposable article according to claim 1, wherein the predetermined portion is a part facing a drive head of the medical device.

4. The disposable article according to claim 3, wherein the part is a syringe plunger plate that faces a membrane disk of the medical device.

5. The disposable article according claim 1, wherein the machine-readable identification feature is selected from the group consisting of:
a shape-based coding;
an electronic-physical measurand; and
an optical code.

6. A medical device for automated administration of medication to a patient, the medical device comprising:
a recognition device for identifying a disposable article, the recognition device comprising a sensor arrangement which is set up to detect a machine-readable recognition feature of the disposable article;
a control device connected to the sensor arrangement, the control device configured to identify the machine-readable recognition feature of the disposable article based on recorded information; and
a drive unit controlled by the control device to automatically administer the medication to the patient according to recognized information of the disposable article.

7. The medical device according to claim 6, further comprising an activator for activating and deactivating the recognition device.

8. The medical device according to claim 6, wherein the drive unit comprises the recognition device.

9. The medical device according to claim 6, wherein the recognition device is part of or attached to the drive unit.

10. The medical device according to claim 6, further comprising a support configured to support the disposable article in the medical device, wherein the support comprises the recognition device.

11. The medical device according to claim 6, wherein the drive unit comprises a sensor signal opening.

12. The medical device according to claim 6, wherein the recognition device comprises an optical conductor connected to the sensor arrangement.

13. The medical device according to claim 6, wherein the recognition device is arranged for centrally detecting the machine-readable recognition feature.

14. The medical device according to claim 6, further comprising a shading device for at least partially shading the machine-readable recognition feature.

15. The medical device according to claim 6, wherein the sensor arrangement is configured to detect the machine-readable recognition feature.

16. The medical device according to claim 15, wherein the sensor arrangement is configured to detect at least one of:
a shape-based coding;
an electronic-physical measurand; or
an optical code.

17. A system comprising:
a storage unit for storing medical data in a database;
a medical device comprising a communication interface for transmitting the medical data; and
a disposable medical article for a medical device, the disposable medical article comprising a machine-readable identification feature, the machine-readable identification feature being a feature of the disposable medical article or being attached to a predetermined partial section of the disposable medical article in a detachable or non-detachable manner.

18. The system according to claim 17, further comprising a control unit configured to, based on the disposable medical article:
filter or limit a medication selection option in the database;
filter or limit a selection option of another single-use article; and/or
issue an alarm or prevent a therapy from starting when the therapy of the medical device with a predetermined combination of drug or drug concentration to the disposable medical article is set.

19. A method for recognizing feature-dependent drug administration in a medical system, the method comprising the steps of:
inserting a disposable article comprising a machine-readable identification feature into a medical device;
recognizing the disposable article based on the machine-readable identification feature of the disposable article;
identifying the machine-readable identification feature of the disposable article based on recorded information; and
controlling a drive unit for automated administration of a medicament in accordance with recognized information of the disposable article.
